(19) United States
(12) Patent Application Publication
(10) Pub. No.: US 2025/0102498 A1
(43) Pub. Date: Mar. 27, 2025

(54) COMPOSITIONS OF LIPOPHILIC ANCHORS AND THE USE THEREOF

(71) Applicant: The Hong Kong University of Science and Technology, Hong Kong (CN)

(72) Inventors: Ying CHAU, Hong Kong (CN); Chi Ming Laurence LAU, Hong Kong (CN); Jin Teng CHUNG, Hong Kong (CN); Manisha Sandupama ABESEKARA, Hong Kong (CN)

(21) Appl. No.: 18/565,591

(22) PCT Filed: Jul. 13, 2023
(86) PCT No.: PCT/IB2023/000557

§ 371 Date: Nov. 30, 2023

Related U.S. Application Data
(60) Provisional application No. 63/389,897, filed on Jul. 17, 2022.

(51) Int. Cl. G01N 33/543; B01J 31/02; G01N 33/92

(52) U.S. Cl. CPC G01N 33/5432; B01J 31/0237; G01N 33/54353; G01N 33/92; G01N 2405/00

(57) ABSTRACT

A lipophilic anchor includes a conjugation domain linked via a linker domain to an anchor domain having a plurality of hydrophobic tails. This lipophilic anchor allows deposition of at least one lipophilic target molecule with affinity for the anchor domain on a hydrophilic surface conjugated to the conjugation domain. The lipophilic target molecule coated hydrophilic substrate has the conjugation domain of the lipophilic anchor bound to the hydrophilic surface of a substrate and lipophilic target molecules are non-covalently bound to the anchor domain.

### CROSS-REFERENCE TO RELATED APPLICATION

This application claims the benefit of U.S. Provisional Application Ser. No. 63/389,897, filed Jul. 17, 2022, which is hereby incorporated by reference in its entirety including any tables, figures, or drawings.

### BACKGROUND OF THE INVENTION

A surface can be functionalized with target molecules by coating. For example, coating phospholipids can be placed on the surface of a material for implantation in the body to improve the material's biocompatibility. Another example of a functionalized surface is for generating an ELISA assay, where a certain type of protein molecules is coated on a polystyrene surface, to enable the surface for specifically binding molecules having a binding affinity with the protein. This coating approach can be simple and unspecific, such as a coating with hydrophobic domains where the target molecules attach to the hydrophobic surface by hydrophobic interactions. Such interactions are stable in an aqueous environment. However, this coating approach is only applicable with hydrophobic surfaces.

For a hydrophilic surface, a target molecule needs to be coated using other interactions, for example, ionic interactions between counter charges or by chemical conjugations. The ionic interactions are often weak in an aqueous environment and is often sensitive to charge change (e.g. due to pH change), and the conjugation can be complicated and may require prior chemical modification of the target molecules or may interfere with the normal function of these molecules. Accordingly, a versatile and effective approach to coat target molecules on a hydrophilic surface by simple hydrophobic interactions is needed.

### BRIEF SUMMARY OF THE INVENTION

An embodiment is directed to a lipophilic anchor that includes a conjugation domain linked via a linker domain to a anchor domain having a plurality of hydrophobic tails. This lipophilic anchor allows deposition of at least one lipophilic target molecule with affinity for the anchor domain on a hydrophilic surface conjugated to the conjugation domain. The conjugation domain is derived from a molecule having at least one functional group selected from: acrylate; methacrylate; maleimide; vinylsulfone; aldehyde; acrylamide; vinyl; thiol; amine; alkylamine: hydroxy; derivatives thereof, and any combination thereof. The hydrophobic tails can be at least one saturated, mono-unsaturated, or polyunsaturated hydrocarbons, where the hydrocarbons can be linear, mono-branched, poly-branched, cyclic, polycyclic, or any combination thereof which can be 6 to 20 carbon atoms, for example, 8 to 14 carbon atoms, that are optionally interrupted with one or two oxygen atoms, sulfur atoms, or a combination thereof. The anchor domain is derived from a second molecule having 1 to 6 hydrophobic tails, for example 1 and/or 2 and/or 3 hydrophobic tails and a complementary functionality to the functionality of the first molecule. In an embodiment the at least one functional group is an acrylate or a thiol and the complimentary functionality can be a thiol or an acrylate, respectively. The linker domain is a structural unit generated upon coupling the first molecule to the second molecule to generate the conjugation domain and anchor domain.

Another embodiment is directed to lipophilic target molecule coated hydrophilic substrate that has a multiplicity of reaction residues of the functionality of the lipophilic anchor disclosed above and a complimentary functionality to the functionality of the conjugation domain of the lipophobic anchor on at least a hydrophilic surface of the hydrophilic substrate and has a multiplicity of lipophilic target molecules non-covalently bound to the plurality of hydrophobic tails of the anchor domain. The hydrophilic surface can be a hydrophilic polymer such as a hydrogel. In embodiments the hydrophilic substrate is a hydrogel, for example a dextran-based hydrogel. In embodiments the multiplicity of lipophilic target molecules are selected from at least one biomolecule, such as phosphatidylcholines (PC), phosphatidylethanolamines (PE), phosphatidylglycerols (PG), phosphatidylserines (PS), sterols, saccharolipids, lipopeptides, lipoproteins, crude membrane components extracted from mammalian cells. The lipophilic target molecule can be a lipid bilayer and the lipophilic target molecule coated hydrophilic substrate in the form of a cell mimic.

Another embodiment is directed to a method of preparing the lipophilic anchor where a first molecule comprising a multiplicity of functional groups selected from: acrylate; methacrylate; maleimide; vinylsulfone; aldehyde; acrylamide; vinyl; thiol; amine; alkylamine: hydroxy; derivatives thereof, and any combination thereof and a second molecule comprising at least one hydrophobic tail selected from a saturated, mono-unsaturated, and polyunsaturated hydrocarbons and a multiplicity of complimentary functional groups are combined. Optionally, a solvent and/or a catalyst or an initiator can be included such that one can isolate the lipophilic anchor with the conjugation domain from the first molecule, the anchor domain from the second molecule and the linker domain resulting from a reaction between the functional groups and the complimentary functional groups.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings herein, of which:

FIG. 1 illustrates the design of lipophilic anchors that include a conjugation domain, a linker domain, and an anchor domain, where the conjugation domain has one or more functional groups to conjugate on the hydrophilic surface and the anchor domain includes two to four aliphatic units that adsorb and stabilize target molecules having lipophilic domains via hydrophobic interactions.

FIG. 2 shows some reagents useful for synthesis of the lipophilic anchors.

FIG. 3 illustrates the process of coating a stable lipid layer to a lipophilic anchor modified hydrophilic surface.

FIG. 4 shows the chemical structure of exemplary lipophilic anchor synthesized using methods according to embodiments.

FIG. 5 shows structures of a mono lipid and two exemplary lipophilic anchors synthesized using methods, according to embodiments.

FIG. 6 shows structures and ¹H NMR spectra of six exemplary lipophilic anchors synthesized using methods, according to embodiments.

FIG. 7 shows structures and ¹H NMR spectra of six exemplary lipophilic anchors synthesized using methods, according to embodiments.

FIG. 8 shows the confocal images of different cell membrane extracts coated on lipophilic anchor (3O-2S12), according to an embodiment, modified HMPs.

FIG. 9 shows confocal images comparing sonication or vortexing at different treatment times to coat cell membrane vesicles on lipophilic anchor (3O-2S12), according to embodiments, modified HMPs.

FIG. 10 shows confocal images of different lipids (DLPC, DSPC, POPG, POPC and cell membrane crude extracts) coated on hydrogel micro particles (HMPs) modified with a mono lipophobic anchor and exemplary lipophilic anchors, according to embodiments.

FIG. 11 shows the confocal images over time after mixing lipophilic anchor decorated HMPs with POPC liposome.

FIG. 12A shows confocal images of POPC coated HMPs with different lipophilic anchors varied in number of hydrophobic tails, and length of hydrophobic tails, according to embodiments.

FIG. 12B shows confocal images of DSPC coated HMPs with different lipophilic anchors, according to embodiments, over time for HMPs after incubation in pH 7.4 PBS with 0.05 w/v % NaN₃ for about 37° C.

FIG. 13 shows the confocal images of different POPC liposome formulations comprising different Biotin-PE contents coated on lipophilic anchor (3O-2S12 and 40-3S12), according to embodiments, modified HMPs.

FIG. 14 shows the confocal images of cell membrane vesicles coated lipophilic anchor (3O-2S12), according to embodiments, modified HMPs stored in cell culture medium at room temperature and 37° C. over 10 days.

FIG. 15 shows the confocal images of POPC coated HMPs with different lipophilic anchors, according to embodiments, over time for HMPs incubated in MEM culture medium with 20% FBS and 0.05 w/v % NaN₃, at about 37° C.

FIG. 16 shows the confocal images of POPC coated HMPs (hydrolysable) with different lipophilic anchors, according to embodiments, over time for HMPs incubated in respectively in RPMI-1640 culture medium with 10% FBS and 1% P/S, or in PBS, both at about 37° C. with 5% CO₂ supply. The signal intensity is also quantified and plotted over time.

### DETAILED DISCLOSURE OF THE INVENTION

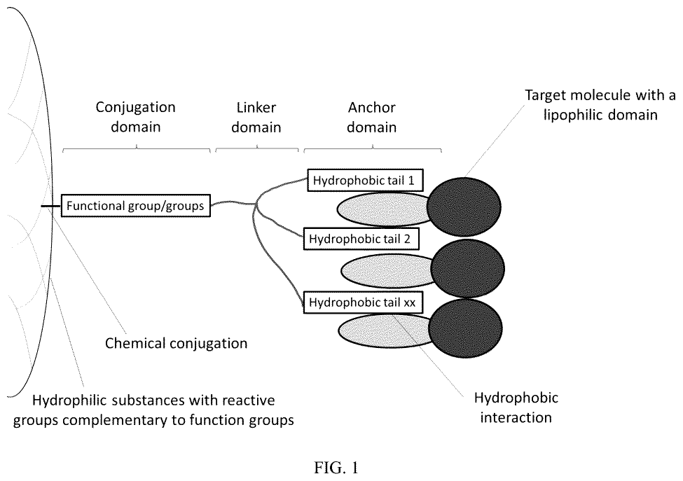

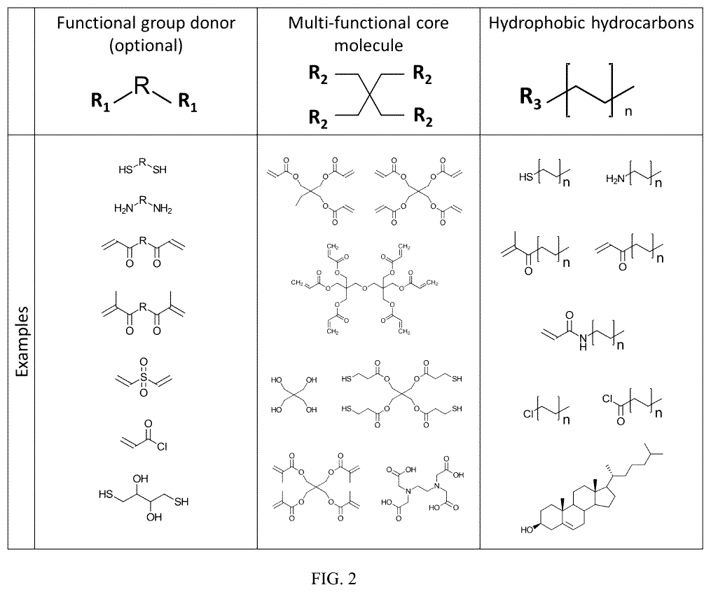

In an embodiment, a lipophilic anchor for adsorbing and stabilizing one or more target molecules having a lipophilic domain allows the deposition of the target molecules on a hydrophilic surface. As shown in FIG. 1, The lipophilic anchors has a modular design of a conjugation domain coupled to an anchor domain by a linker domain. FIG. 2 shows non-limiting exemplary reagents that can be used to synthesize the lipophilic anchors, according to embodiments. The conjugation domain serves to chemically conjugate the lipophilic anchor to a hydrophilic surface. The anchor domain assembles with a target molecule via hydrophobic interactions. The linker domain links the conjugation and anchor domains. One or more of: the conjugation domain; linker domain; and anchor domain is optionally cleavable from the bound anchor by hydrolysis, or enzymatic degradation, or other mechanism.

In embodiments, the hydrophilic surface is on a hydrophilic substrate that readily wets with water, and can be a glass, metal, biological tissue, and hydrogel surface. The substrate may be flat or have curvature having any regular or irregular shape. In embodiments the hydrophilic surface can be that of a hydrophilic spherical particle including a hydrogel, allowing formation of a cell mimic, an “artificial cell” with the attachment of a lipid layer or lipid bilayer membrane to the anchor domain of the lipophilic anchor. Such cell mimics may be used for immune cell therapy. The stable coating of the lipid shell on the hydrogel particles allows a fluidity of the cell mimic for interaction with a biological medium. Signaling proteins can be docked on the membrane allowing a mimic of the native interaction between natural cells.

The hydrophilic surface has a multiplicity of reactive groups for bonding to one or more complementary functional groups on the lipophilic anchor, where the functional groups may be selected from, but are not limited to: acrylate; methacrylate; maleimide; vinylsulfone; aldehyde; acrylamide; vinyl; thiol; amine; derivatives thereof; and any combinations thereof. The conjugation domain comprises at least one functional group selected from, but not limited to: acrylate; methacrylate; maleimide; vinylsulfone; aldehyde; acrylamide; vinyl; thiol; amine; alkylamine: hydroxy; derivatives thereof, and any combination thereof.

In embodiments, the linker domain is a structural unit that branches between one or more functional groups in the conjugation domain to a plurality of hydrophobic tails in the anchor domain. These two or more hydrophilic tails can be saturated, mono-unsaturated, or polyunsaturated hydrocarbons that can be linear, mono-branched, poly-branched, cyclic, polycyclic, or any combination thereof. To achieve the desired hydrophobic interactions the hydrophobic tails can be of 6 to 20 carbon atoms that can be uninterrupted or interrupted with one or two oxygen atom, sulfur atom, or a combination thereof. The hydrophobic tail can include a hydrocarbon that is 8 to 14 carbon atoms. The anchor domain can include two, three, four, or five hydrophobic tails to adsorb and stabilize target molecules with lipophilic domains via hydrophobic interactions.

Although not limited to, exemplary embodiments, the substrate providing the hydrophilic surface is a hydrogel microparticle (HMP). The HMP can be a mixture of particles having a diameter of 0.1 to 10,000 micrometer, for example, the mixture can be 0.1 to 1 micrometer, 1-1000 micrometer, or 1-10 millimeter, with a narrow or broad distribution of diameters. The HMP can be a polymeric gel formed from one or mor polymer precursors having a weight averaged molecular weight of about 5 kDa to about 3000 kDa. As used herein a polymer can be a homopolymer, copolymer, or terpolymer, where the co- or terpolymer can be random, block, linear, branched, or hyperbranched. Where the precursor can have complementary functional groups for attachment to their compliment in the conjugation domain where the precursor has, for example, but not limited to, about 1% to 30% of the polymers repeating units. The complementary functional groups can be randomly or non-randomly dispersed and can be homogeneously situated within the HMP or concentrated on or near the external surface of the HMP. The HMPs forming precursors consist hydrophilic monomers with reactive functional groups selected from acrylate, methacrylate, maleimide, vinylsulfone, aldehyde, acrylamide, vinyl, thiol, amine, alkyl amine, hydroxy, derivative thereof, and any combinations thereof. The HMPs can be formed prior to modification with the lipid anchor or the HMPs can be formed and simultaneously modified with the lipid anchor.

The lipophilic anchors on a hydrophilic surface increases the amount of adsorbed target molecules from an aqueous environment. The amount of target molecules being adsorbed on a hydrophilic surface in an aqueous environment is varied by varying the modification density of lipophilic anchors.

In some embodiments, the modification density of lipophilic anchors on a hydrophilic surface is adjustable by changing the amount of complementary reactive functional groups on the hydrophilic surface, or inside the substance providing the hydrophilic surface. By incorporating one or more specific types of lipophilic anchors, the efficiency of adsorbing specific target molecules or desired combinations of target molecules from the aqueous environment on the hydrophilic surface.

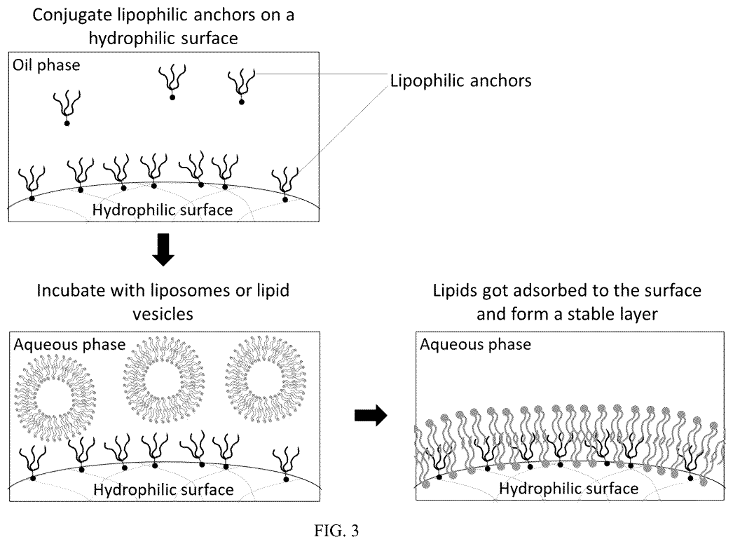

In embodiments, the target molecules of the lipophilic domains are selected from phosphatidylcholines (PC), phosphatidylethanolamines (PE), phosphatidylglycerols (PG), phosphatidylserines (PS), sterols, dye molecules, fluorescent dye molecules, saccharolipids, lipopeptides, lipoproteins, and crude membrane components extracted from mammalian cells. FIG. 3

The inclusion of the lipophilic anchors on the hydrophilic surface prolongs the adhesion time of target molecules being adsorbed depending upon the structure and quantity of the lipophilic anchors. The adhesion time can range from 1-24 hours, 1-7 days, 1-4 weeks, or 1-12 months in an aqueous environment. The aqueous environment can be a physiological or mammalian cells cocultures. In some embodiments, the adhesion time can be based on the degradation rate of cleavable groups embedded within the lipophilic anchors or cleavable groups between the hydrophilic surface and the lipophilic anchors.

The cell mimics can be used to modulate functions of other immune cells, for example production of CAR-T cells, or as a biomaterial platform for vaccine applications. The lipophilic anchor domain stabilized lipid coated particles can be used as a platform for high throughput lipopeptide/lipoprotein signal screening. The lipid surface stabilized by the lipid anchor on a hydrophilic surface can be employed can be employed to establish a lipophilic anchor library for the systematically analyze of or screen for the effects of different anchor structures and the resulting membrane stability in a physiological environment, especially in cell culture media with proteins. The lipophobic anchors can allow the coating of liposomes formulated by synthetic lipids or by cell membrane extracts.

All publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. The numbers for amounts, temperature, and other specific quantities are accurate within normal experimental errors and deviations. Unless indicated otherwise, parts are parts by weight, molecular weight is weight average molecular weight, temperature is in degrees Celsius, and pressure is at or near atmospheric. Standard abbreviations used include: bp, base pair(s); kb, kilobase(s); pl, picoliter(s); s, sec, or second(s); min or minute(s); h, hr, or hour(s); aa, amino acid(s); nt, nucleotide(s); i.m., intramuscular (ly); i.p., intraperitoneal (ly); s.c., subcutaneous (ly); and the like.

### MATERIALS AND METHODS

### Preparation of Hydrogel Microparticles (HMPs) Using Batch Emulsion or Droplet-Based Microfluidic Approach

The hydrogel forming precursors were prepared using methods disclosed in Lau, Chi Ming Laurence, et al. “Controllable multi-phase protein release from in-situ hydrolysable hydrogel.” Journal of Controlled Release 335 (2021): 75-85. Dextran-based hydrogel microparticles (HMPs) were prepared using the microfluidic flow focusing methods disclosed in Chung, Casper HY, et al. “Droplet-Based Microfluidic Synthesis of Hydrogel Microparticles via Click Chemistry-Based Cross-Linking for the Controlled Release of Proteins.” ACS Applied Biomaterials 4.8 (2021): 6186-6194, or using porous membrane aided batch emulsion.

Microfluidic flow focusing method: Hydrogel forming precursors were dissolved in a buffer with pH ranged from 5-6, or 6-7, or 7-8, and subsequently mixed and injected to n-heptane or mineral oil as the continuous phase, using 1-4% v/v SPAN80, a mixture of SPAN80/TWEEN80, or EM90 as the surfactants. Droplets were incubated at room temperature for at least one hour to allow gelation. Amounts of unreacted thiol groups remaining in the HMPs, were confirmed by adding HMPs into Ellman's reagent.

Porous membrane aided batch emulsion method: Hydrogel-forming precursors and oil phase were prepared in the same way as described in the microfluidic flow focusing method. The materials to be encapsulated, such as recombinant proteins, nucleic acids, nanoparticles, or microparticles, are added to one or both precursor solutions to create a mixture of solution or suspension. Prior to mixing, the hydrogel-forming precursors are kept in an ice bath. After thorough mixing, the aqueous phase mixture is transferred to the oil phase. The volume of the aqueous phase is less than 30% v/v of the oil phase. The aqueous droplets are formed by passing the aqueous phase mixture and the oil phase through a porous membrane multiple time (2-30 times). The resulting emulsion is incubated at room temperature for at least one hour to allow for gelation. HMPs contain a certain amount of unreacted thiol groups, which can be confirmed by a positive result from adding HMPs to Ellman's reagent.

### Preparation of Lipophilic Anchors

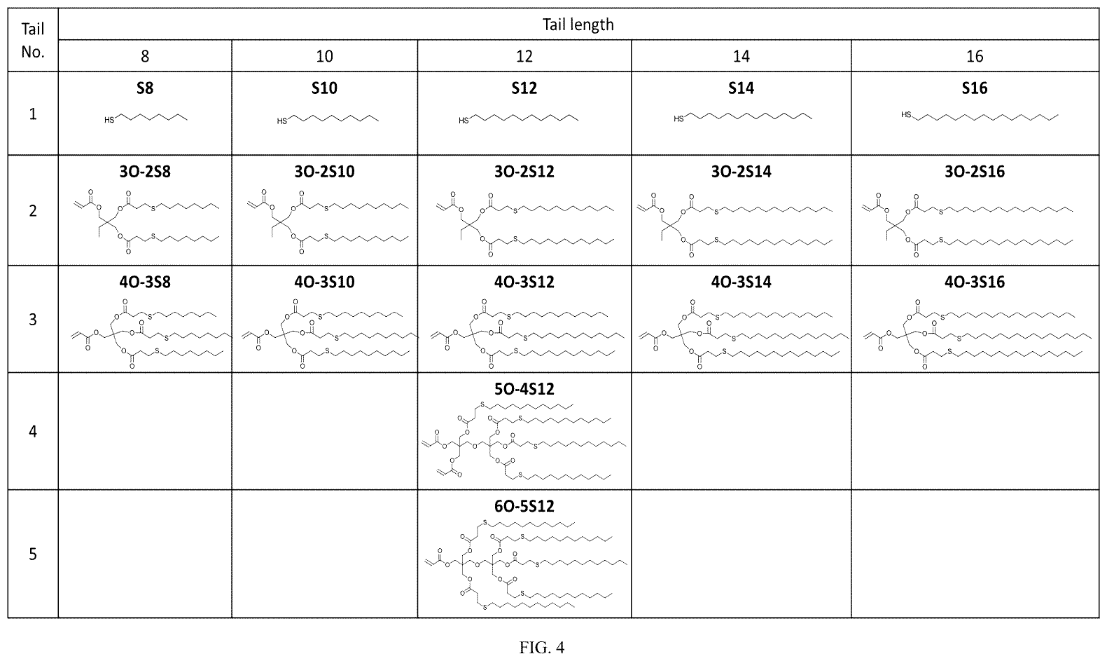

Exemplary lipophilic anchors synthesized by reacting a series of thiolated hydrocarbons with varied length (8-16 carbons) with various multi-acrylate core molecules are illustrated in FIG. 4. The exemplary multi-acrylate core molecules: trimethylolpropane triacrylate (3O); pentaerythritol tetraacrylate (4O); or dipentaerythritol hexaacrylate (6O) were dissolved in dimethylformamide (DMF) to a concentration of about 0.05-0.2 M, and added with thiolated hydrocarbons (e.g 1-octanethiol (S8), 1-decanethiol (S10), 1-dodecanethiol (S12), 1-tetradecanethiol (S14), 1-hexadecanethiol (S16)) at an equivalent feeding ratio to react using a desired number, for example, 2, 3, 4, or 5 thiolated hydrocarbons with the acrylates of the core molecule. An organic base, such as, but not limited to a trialkylamine, for example, triethylamine, was included as the catalyst.

A typical reaction was conducted at a temperature of about 15-50° C. for about 4-24 hours. If lipophilic anchors precipitate during the reaction, for example, 30-2S16, 40-3S12, 40-3S14, 40-3S16, 60-4S12, 60-5S12, 2-6 mL of chloroform was added to the reaction mixture to dissolve the precipitations. The reaction endpoint was determined by detecting minimum residual amount of free thiol groups in the reaction mixture using Ellman's reagent. The lipophilic anchors were extracted using chloroform:methanol (3:1-2:1) and washed 3-10 times with dilute HCl (0.01-0.05M) and water. Residual chloroform was removed by purging with nitrogen gas and vacuum drying to yield crude products as a viscous liquid or a waxy solid.

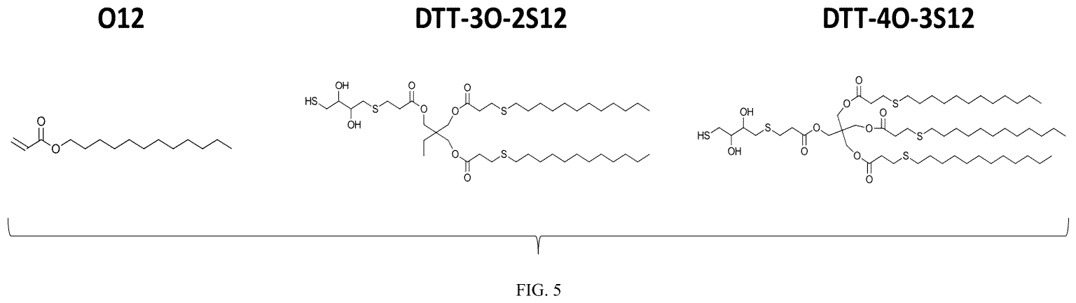

In some embodiments, an optional functional group donor (e.g DL-dithiothreitol (DTT)) was conjugated to the free functional group (e.g acrylate) to change the functional group for crosslinking, and adjust the hydrophilic-lipophilic balance (HLB) value of the lipophilic anchors, such as DTT-30-2S12 and DTT-40-3S12, as shown in FIG. 5.

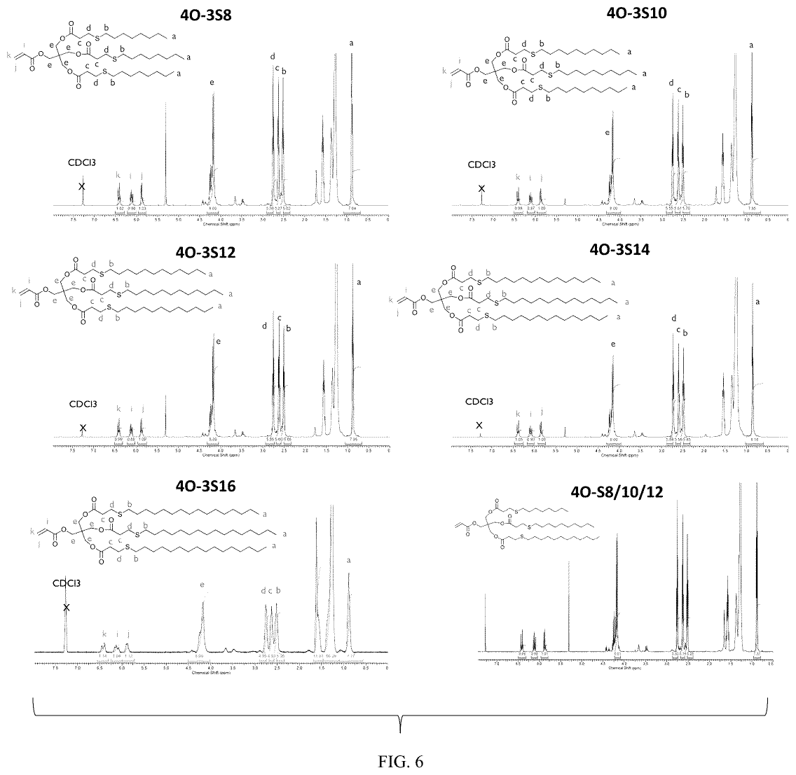

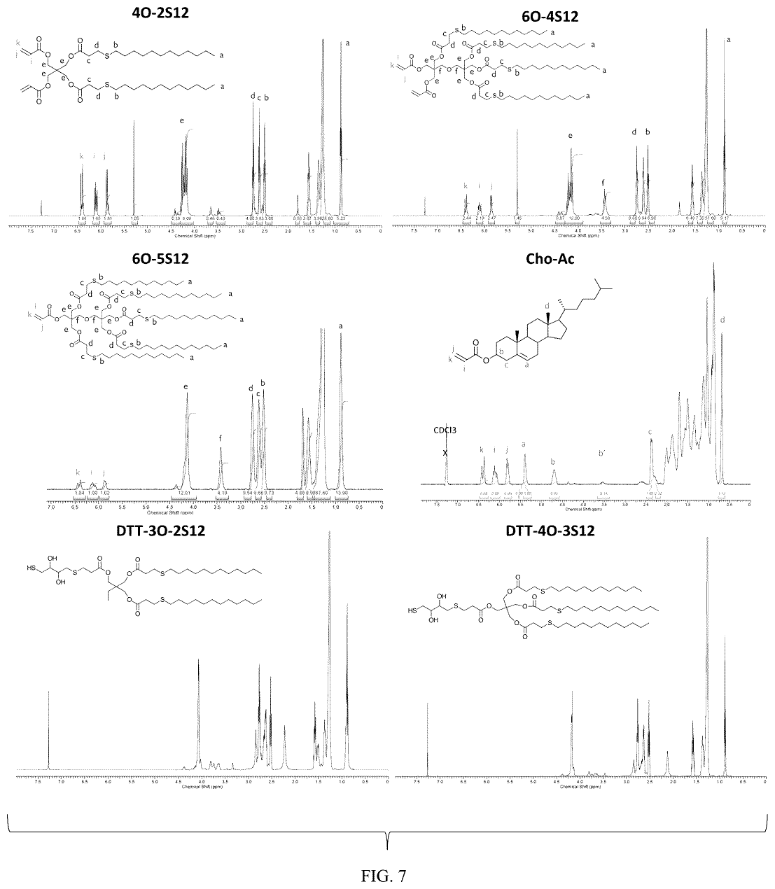

The products were characterized using 1H NMR using d-chloroform as solvent, as in FIGS. 6-7. Some representative synthesis schemes are shown in Table 1, below.

TABLE 1
Reagents and Solvent for Various Lipophilic Anchor Compositions

| | Core | | lipid | lipid | | |
|---|---|---|---|---|---|---|
| Lipophilic | molecule | anchor | tail | tail | | |
| anchor | No. | lipid | No. | vol | TEA | DMF |
| code | mmol | tails | mmol | (μL) | (μL) | (mL) |
| 3O-2S8 | 0.241 | 2 | 0.482 | 86 | 67 | 4.8 |
| 3O-2S10 | 0.281 | 2 | 0.561 | 123 | 78 | 5.6 |
| 3O-2S12 | 0.278 | 2 | 0.555 | 140 | 77 | 5.6 |
| 3O-2S14 | 0.276 | 2 | 0.552 | 154 | 77 | 5.5 |
| 3O-2S16 | 0.348 | 2 | 0.697 | 218 | 97 | 7.0 |
| 4O-3S8 | 0.287 | 3 | 0.860 | 153 | 120 | 5.7 |
| 4O-3S10 | 0.324 | 3 | 0.971 | 212 | 135 | 6.5 |
| 4O-3S12 | 0.285 | 3 | 0.854 | 215 | 119 | 5.7 |
| 4O-3S14 | 0.292 | 3 | 0.875 | 245 | 122 | 5.8 |
| 4O-3S16 | 0.299 | 3 | 0.897 | 281 | 125 | 6.0 |
| 4O-2S12 | 0.92 | 2 | 1.84 | 465 | 258 | 9.2 |
| 6O-4S12 | 0.39 | 4 | 1.17 | 298 | 328 | 7.8 |
| 6O-5S12 | 0.33 | 5 | 1.36 | 342 | 280 | 6.6 |

### Conjugating Lipophilic Anchors on HMPs Surface

In some embodiments, preformed HMPs containing free vinyl sulfone, acrylate, methacrylate, maleimide, or thiol groups were washed three times with excess n-heptane or DMF and transferred to a lipophilic anchor solution using a nonpolar organic solvent such as n-heptane or a polar organic solvent such as DMF. Some HMPs with reactive vinyl sulfone, or acrylate, or methacrylate, or maleimide were modified with thiol-containing anchors, while some HMPs with free thiols were modified with acrylate-containing, or vinyl sulfone containing anchors. A lipophilic anchor solution was prepared by dissolving anchors in n-heptane, or DMF to a concentration of about 1-8 v/v %. The mixture was homogenized by pipetting and vortex. For anchors having lipophilic anchors that do not dissolve in DMF under ambient temperature, a controlled amount of DCM, or chloroform was added to solubilize the anchors. Triethylamine (TEA) was added at 0.05-0.2M to catalyze the reaction. The HMP-anchor mixture was incubated for 2-36 hours with shaking at ambient temperature in a sealed glass container to allow conjugation of lipophilic anchors on the HMPs surface. Unconjugated lipophilic anchors were sequentially washed with excess DMF and ethanol multiple times. Anchor modified HMPs were preserved in ethanol.

### Coating of Synthetic Lipids and Hydrophobic Dyes on the Surface of Lipophilic Anchors Modified HMPs

Synthetic or natural product extracted phospholipids including 12:0 PC (DLPC), 16:0-18:1 PC (POPC), 18:0 PC (DSPC), 16:0-18:1 PG (POPG), 18:1 Liss Rhod PE (LissRhoPE), 18:1 Biotinyl Cap PE (biotinPE) were coated on lipophilic anchor modified surface of HMPs. LissRhoPE was added at 0.1% to all liposome formulations as a color indicator.

Liposomes comprising DLPC:LissRhoPE 99.9:0.1; POPC:LissRhoPE 99.9:0.1 were prepared using ethanol injection method. In brief, DLPC and LissRhoPE were dissolved in ethanol separately, then mixed at molar ratio of 99.9:0.1. The mixture was rapidly injected to PBS of at least 10 times larger volume to a final lipid concentration of 1-2 mM, vortex mixed, then vacuum dried to remove the ethanol. POPC:LissRhoPE liposome was prepared using the same method.

Liposomes comprising DSPC:LissRhoPE 99.9:0.1; POPG:LissRhoPE 99.9:0.1 were prepared using film hydration method. Separately, DLPC or POPG and LissRhoPE were dissolved in chloroform and ethanol, respectively. Then mixed at molar ratio of 99.9:0.1. Chloroform and ethanol were evaporated in a vacuum with heating to about 60° C. to form a viscous thin film. The film was rehydrated with pre-warmed PBS to have a final concentration of 1-2 mM and vortex mixed to form liposomes.

To coat lipid layer on anchor modified HMP, liposomes suspension was mixed with HMPs, homogenized by vortex mixing, then incubated at ambient temperature for 1-48 hours. In some cases, brief sonication and repeated freeze-thaw cycles were applied to facilitate lipid coating. Afterwards, uncoated liposomes were washed with excess PBS, and stored in pH 7.4 PBS at 4° C.

### Coating of Cell Membrane Extracts on the Surface of Lipophilic Anchors Modified HMPs

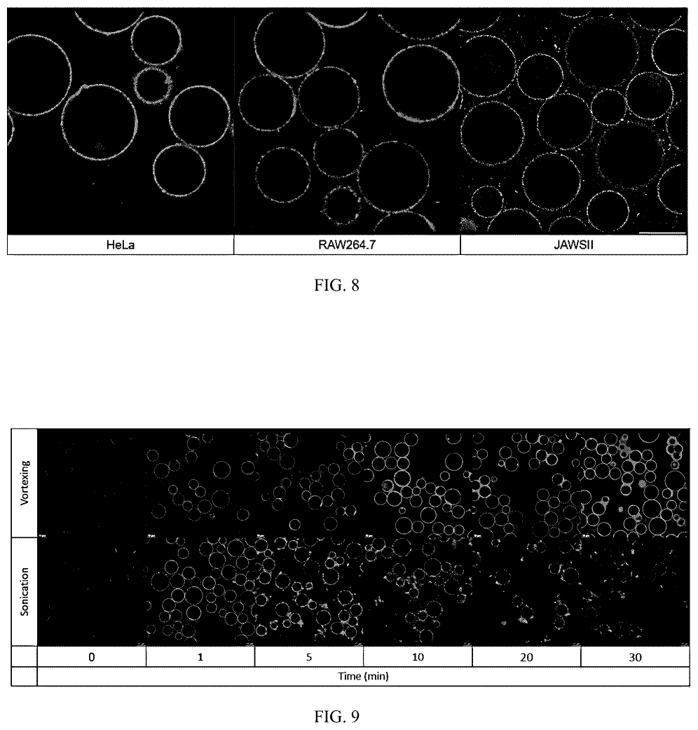

Crude membrane components were extracted from three different mammalian cells, namely HeLa, RAW264.7 and JAWS II using a method modified from that disclosed in Liu, L. et al. Cell membrane coating integrity affects the internalization mechanism of biomimetic nanoparticles. Nature Communications 12, (2021). Extracted cell membranes were stored in PBS buffer (pH 7.4) with protease and phosphatase inhibitor cocktail prior to use. Prior to coating, nano-size cell membrane vesicles (below 100 nm) were prepared by extruding cell membranes through a polycarbonate membrane (pore size 0.100 μm) a series of 11 times. For fluorescence imaging experiments, 1% (w/w) LissRhoPE was added to the extruded mixture. All the cell membranes were able to adsorb into lipophilic anchor modified HMPs, as indicated in FIG. 8.

Vortexing and sonication were evaluated to coat 40-3S12 HMPs with cell membrane vesicles. Both vortexing and sonication facilitate the adsorption of cell membrane vesicles on the 40-3S12 HMPs, as indicated in FIG. 9, however, sonication for more than 5 minutes resulted in aggregation of vesicles affecting consistency of the coating. Membrane coating content can be increased by increasing the vortexing time, although aggregation of HMPs appears to occur at longer time intervals.

### Absorption of Synthetic Lipids and Cell Membrane Extracts on the Surface of Lipophilic Anchors Modified HMPs

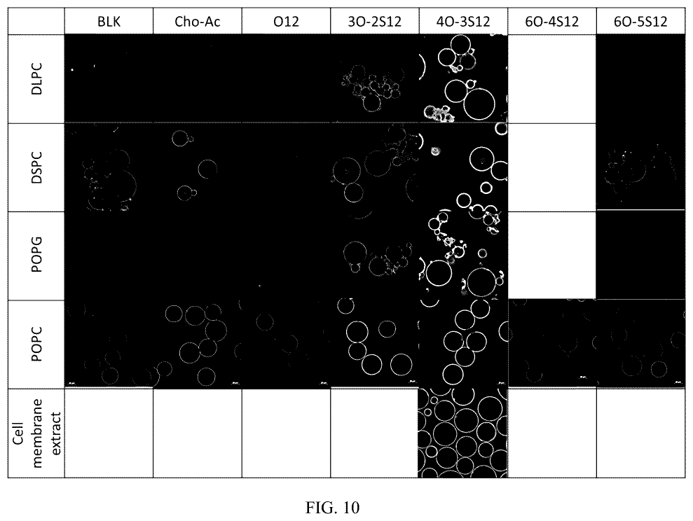

The amount of synthetic lipids (e.g DLPC, DSPC, POPG, POPC), and mammalian cell membrane contents (e.g HeLa, RAW264.7 and JAWS II) adsorbed onto the HMPs surface was estimated based on the fluorescent signals of LissRhoPE at 561/594 nm using confocal microscopy images, as illustrated in FIG. 10, employing common imaging settings for all examples.

The lipophilic anchor 40-3S12 exhibited a universal high capability to adsorb different types of synthetic lipids being tested, such as DLPC, DSPC, POPG, POPC. Anchor 3-2S12 had comparable performance to adsorb POPC, but less effective for DLPC, DSPC and POPG adsorption. Further increasing the number of hydrophobic tails attenuated the lipid adsorption capability. Lipophilic anchors having 2 and 3 hydrophobic tails in the anchor domain demonstrated the highest lipid adsorption, with 3 hydrophobic tails demonstrating a higher versatility for different types of lipids.

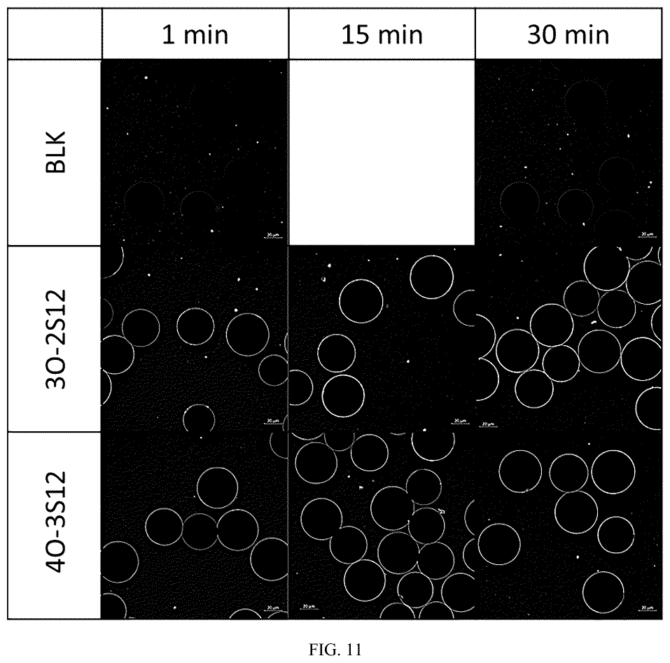

The adsorption POPC from POPC/LissRhoPE 99.9:0.1 liposome was efficient when HMPs were modified with 30-2S12 and 40-3S12 anchors, as shown in FIG. 11. After 1 min that liposome and HMPs were mixed at ambient temperature, distinct POPC/LissRhoPE adsorption was observed from the increased rhodamine signals surrounding the HMPs.

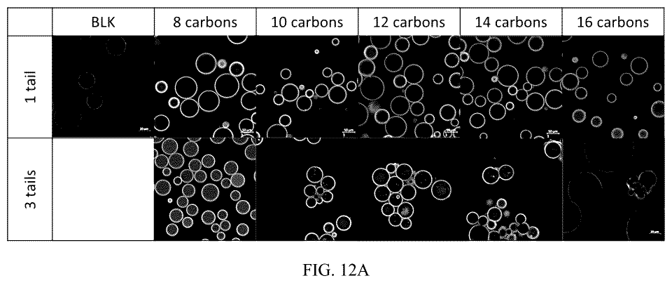

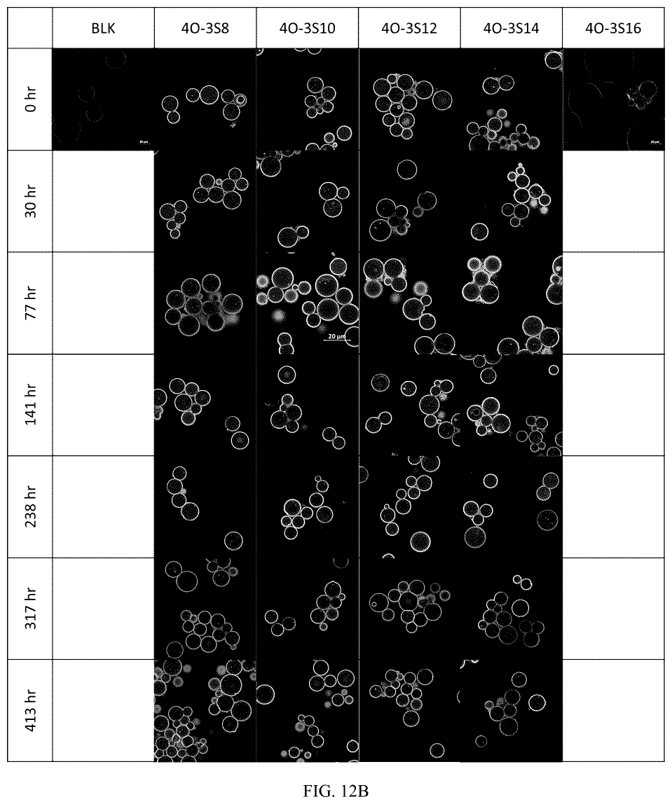

Exemplary lipophilic anchors with single or triple hydrophobic tails of varying lengths from 8 to 16 carbons along the backbone showed different abilities to absorb POPC or DSPC liposomes containing 0.1% Rhodamine PE, as indicated by the amount of lipids coated. Anchors with 8 to 10 carbon tails exhibited the highest lipid absorption for single-tail anchors, as shown in FIG. 12A. Increased tail length was found to decrease the amount of lipids absorbed. For triple-tail anchors, tail lengths from 8 to 14 carbons yielded comparable lipid absorption, but increasing the tail length to 16 carbons reduced the lipid coating capability.

Liposomes with different lipid compositions having varied fractions of different types of lipids were coated on the HMPs modified with 30-2S12 and 40-3S12 according to the following table:

TABLE 2
Liposomes and Lipid Compositions Coated
on HMPs Modified with Lipophilic Anchors

| | Biotin | LissRho | POPC | |
|---|---|---|---|---|
| Liposome | PE fraction | PE fraction | fraction | Liposome |
| formulation | (mol %) | (mol %) | (mol %) | conc. (μM) |
| POPC- | 2.5 | 0.1 | 97.4 | 1000 μM |
| 2.5% Biotin | | | | |
| POPC- | 0.5 | 0.1 | 99.4 | 1000 μM |
| 0.5% Biotin | | | | |
| POPC- | 0.1 | 0.1 | 99.8 | 1000 μM |
| 0.1% Biotin | | | | |

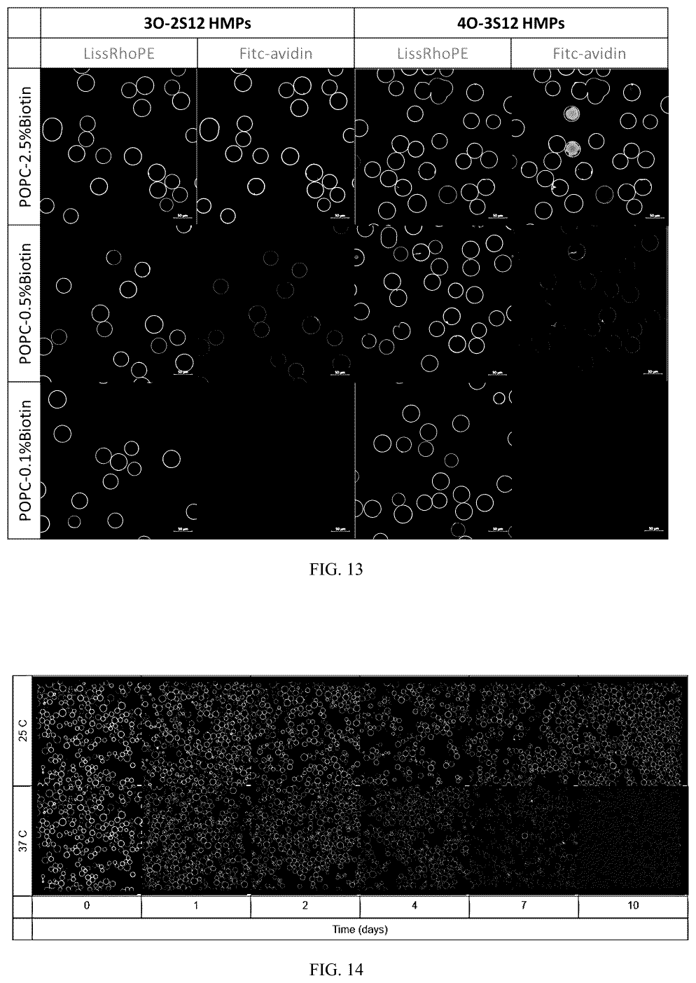

After lipid coating and washing, the HMPs were incubated with 0.1 mg/ml neutravidin, or avidin with/without FITC to graft biotin docking sites onto the HMP surface. Reaction was conducted in 1% BSA/PBS for 60 mins at ambient temperature to avoid unspecific bindings. Unbound avidin was thoroughly washed with PBS. In some embodiments, Fitc labeled avidin was used to quantify the amount of avidin grafted on different HMP formulations differed in biotin contents. Fitc-avidin was expected to bind the exposed biotin group on the HMPs surface, and the amount of Fitc-avidin was expected to correlate with the biotin PE fraction of the liposome formulation coated. Similar LissRhoPE signals of HMPs coated with all formulations indicated that the amount of lipid being coated was similar. The Fitc-avidin signals well correlated with the biotin PE fractions in the corresponding liposome formulations, as shown in FIG. 13.

### Stability of Lipid Coated HMPs in Different Buffers

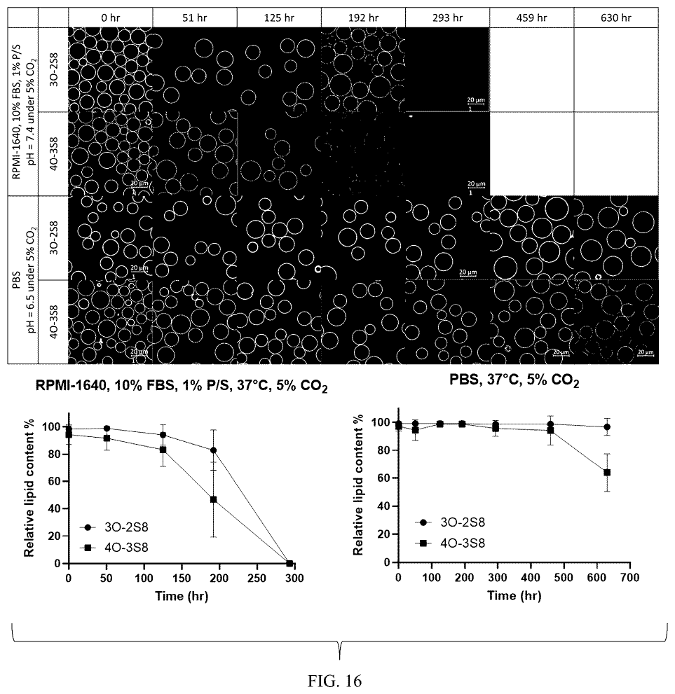

The stability of HMP coated lipid membrane was tested in different buffer systems. In some experiments, lipid coated HMPs were incubated in PBS (pH7.4) with 0.05 w/v % NaN₃, about 37° C. as shown in FIG. 12B. In other experiments, lipid coated HMPs were incubated in Minimum Essential Medium (MEM) with 20% FBS and 0.05 w/v % NaN₃, at about 37° C., as shown in FIG. 15. In other experiments, lipid coated HMPs were incubated in RPMI media with 10% FBS, 1% penicillin-streptomycin, and PBS at about 37° C. respectively with 5% CO₂, as shown in FIG. 16. The stability of lipid layer and cell membrane contents on HMPs surface was indirectly approximated from the residual amount of LissRhoPE at different incubation times. In pH 7.4 PBS at 37° C., a saturated phospholipid, such as DSPC, was stably coated on non-hydrolysable HMP with anchors 40-3S8, 40-3S10, 40-3S12 and 40-3S12, and these anchors all coated DSPC well up to 413 hours (FIG. 12B). In pH 7.4 MEM media with 20% FBS, only 30-2S12 and 40-3S12 anchors were able to maintain a stable DSPC membrane coating up to 19 days, while 30-2S12 demonstrated the best effect for maintaining DSPC up to 29 days as indicated in FIG. 15.

In PBS at 37° C. with 5% CO₂ where the pH was about 6.5, a supply mimicking cell culture environment, more than 90% unsaturated phospholipids, such as POPC, were maintained on hydrolysable HMP with anchors 30-2S8 and 40-3S8 up to 459 hours. However, 40-3S8 HMP starts to lose surface coated POPC after 630 hours, while a 30-2S8 anchor still maintains more than 90% of POPC contents on the HMP surface. In RPMI-1640 medium with 10% FBS under 5% CO₂, the trend was similar to that in PBS. However, the hydrolysable HMP degraded faster in medium due to the higher pH (7.4), and HMP completed dissolved in 293 hours, thus all HMP coated POPC lipids were disassociated, as indicated in FIG. 16.

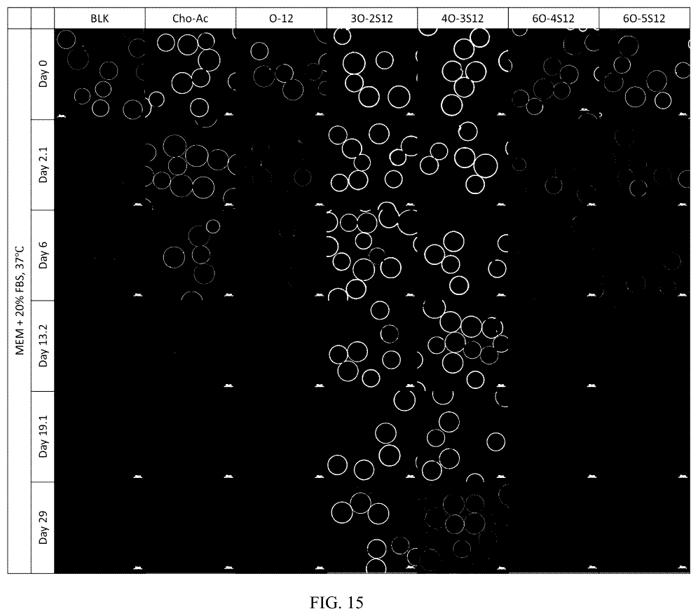

The stability of the cell membrane-coated 40-3S12 modified HMP (by vortex) was studied in Minimum Essential Medium Eagle-Alpha Modification (aMEM) culture medium with 20% FBS and 0.05 w/v % NaN₃, at room temperature and 37° C. for 10 days. The coating was more stable under room temperature, as indicated in FIG. 14. When stability of the coating was checked using HMPs without lipid anchor modification, a disintegrated coating was observed from day 2 for samples under both temperature conditions.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EMBODIMENTS

Embodiment 1. A lipophilic anchor, comprising a conjugation domain linked via a linker domain to a an anchor domain comprising a plurality of hydrophobic tails, whereby at least one lipophilic target molecule with affinity to the anchor domain can be deposited on a hydrophilic surface conjugated to the conjugation domain.

Embodiment 2. The lipophilic anchor according to embodiment 1, wherein the conjugation domain is derived upon reaction of at least one first molecule comprising at least one functional group selected from: acrylate, methacrylate, maleimide, vinylsulfone, aldehyde, acrylamide, vinyl, thiol, amine, alkylamine, hydroxy, derivatives of any of the foregoing, and any combination thereof.

Embodiment 3. The lipophilic anchor according to embodiment 2, wherein the at least one functional group is an acrylate or a thiol.

Embodiment 4. The lipophilic anchor according to embodiment 1, wherein the hydrophobic tails comprises at least one saturated, mono-unsaturated, or polyunsaturated hydrocarbon, where the hydrocarbon is linear, mono-branched, poly-branched, cyclic, polycyclic, or any combination thereof.

Embodiment 5. The lipophilic anchor according to embodiment 4, wherein the hydrocarbon comprises 6 to 20 carbon atoms optionally interrupted with one or two oxygen atoms, sulfur atoms, or a combination thereof.

Embodiment 6. The lipophilic anchor according to claim 4, wherein the hydrocarbon comprises 8 to 14 carbon atoms optionally interrupted with one or two oxygen atoms, sulfur atoms, or a combination thereof.

Embodiment 7. The lipophilic anchor according to embodiment 1, wherein the anchor domain is derived from a second molecule comprising 2 to 6 hydrophobic tails.

Embodiment 8. The lipophilic anchor according to embodiment 6, wherein the second molecule comprises 2 or 3 hydrophobic tails.

Embodiment 9. The lipophilic anchor according to embodiment 1, wherein the linker domain is a structural unit generated upon reaction of the first molecule to the second molecule.

Embodiment 10. A lipophilic target molecule coated hydrophilic substrate, comprising: a multiplicity of reaction residues of the functionality of the lipophilic anchor according to embodiment 1 and a complimentary functionality to the functionality of the conjugation domain of the lipophobic anchor on at least a hydrophilic surface of the hydrophilic substrate; and a multiplicity of lipophilic target molecules non-covalently bound to the plurality of hydrophobic tails of the anchor domain.

Embodiment 11. The lipophilic target molecule coated hydrophilic substrate according to embodiment 9, wherein the hydrophilic surface comprises a hydrophilic polymer.

Embodiment 12. The lipophilic target molecule coated hydrophilic substrate according to embodiment 9, wherein the hydrophilic substrate comprises a hydrogel.

Embodiment 13. The lipophilic target molecule coated hydrophilic substrate according to embodiment 11, wherein the hydrogel is a dextran-based hydrogel.

Embodiment 14. The lipophilic target molecule coated hydrophilic substrate according to embodiment 10, wherein the multiplicity of lipophilic target molecules are selected from at least one biomolecule.

Embodiment 15. The lipophilic target molecule coated hydrophilic substrate according to embodiment 14, wherein the at least one biomolecule is selected from phosphatidylcholines (PC), phosphatidylethanolamines (PE), phosphatidylglycerols (PG), phosphatidylserines (PS), sterols, saccharolipids, lipopeptides, lipoproteins, and crude membrane components extracted from mammalian cells.

Embodiment 16. The lipophilic target molecule coated hydrophilic substrate according to embodiment 10, wherein the lipophilic target molecule is a lipid bilayer and the lipophilic target molecule coated hydrophilic substrate is a cell mimic.

Embodiment 17. A method of preparing the lipophilic anchor according to embodiment 1, comprising:
providing a first molecule comprising a multiplicity of functional groups selected from: acrylate, methacrylate, maleimide, vinylsulfone, aldehyde, acrylamide, vinyl, thiol, amine, alkylamine, hydroxy, derivatives of any of the foregoing, and any combination thereof;
providing a second molecule comprising at least one hydrophobic tail selected from saturated, mono-unsaturated, and polyunsaturated hydrocarbons and a multiplicity of complimentary functional groups;
combining the first molecule and the second molecule and optionally a solvent and/or a catalyst or an initiator; and
isolating the lipophilic anchor comprising the conjugation domain from the first molecule, the anchor domain from the second molecule and the linker domain resulting from a reaction between the functional groups and the complimentary functional groups.

Embodiment 18. The method according to embodiment 17, wherein the functionality is an acrylate, and the complementary functionality is a thiol.

Embodiment 19. The method according to embodiment 17, wherein the optional catalyst is a trialkyl amine.

### REFERENCES

Lau, C. M. Laurence, et al. “Controllable multi-phase protein release from in-situ hydrolysable hydrogel.” Journal of Controlled Release 335 (2021): 75-85.
Chung, C. HY, et al. “Droplet-Based Microfluidic Synthesis of Hydrogel Microparticles via Click Chemistry-Based Cross-Linking for the Controlled Release of Proteins.” ACS Applied Biomaterials 4.8 (2021): 6186-6194.
Liu, L. et al. Cell membrane coating integrity affects the internalization mechanism of biomimetic nanoparticles. Nature Communications 12, (2021). KR101365676 B1 (2014-02-21) “Method of functionalization of solid surface using lipid”.
Karino, T. et al. “Stabilization of hydrophobic domains in hydrogel by intermolecular hydrogen bonds between carboxylic groups at the distal end of alkyl side-chain” Polymer 43 (26) (2002): 7467-7475.
U.S. Pat. No. 7,138,186 B2, (2006-11-21) “Hydrophobic Coatings and Methods”.
U.S. Pat. No. 7,811,555 B2 (2010-10-12) “Tri-Brancehed Biologically Active Copolymer”.
U.S. Pat. No. 9,095,640 B2 (2015-08-04) “Bioactive Implant and Method of Use”.

1. A lipophilic anchor, comprising a conjugation domain linked via a linker domain to an anchor domain comprising a plurality of hydrophobic tails, whereby at least one lipophilic target molecule with affinity to the anchor domain can be deposited on a hydrophilic surface conjugated to the conjugation domain.

2. The lipophilic anchor according to claim 1, wherein the conjugation domain is derived upon reaction of at least one first molecule comprising at least one functional group selected from: acrylate, methacrylate, maleimide, vinylsulfone, aldehyde, acrylamide, vinyl, thiol, amine, alkylamine, hydroxy, derivatives of any of the foregoing, and any combination thereof.

3. The lipophilic anchor according to claim 2, wherein the at least one functional group is an acrylate or a thiol.

4. The lipophilic anchor according to claim 1, wherein the hydrophobic tails comprise at least one saturated, mono-unsaturated, or polyunsaturated hydrocarbon, where the hydrocarbon is linear, mono-branched, poly-branched, cyclic, polycyclic, or any combination thereof.

5. The lipophilic anchor according to claim 4, wherein the hydrocarbon comprises 6 to 20 carbon atoms optionally interrupted with one or two oxygen atoms, sulfur atoms, or a combination thereof.

6. The lipophilic anchor according to claim 4, wherein the hydrocarbon comprises 8 to 14 carbon atoms optionally interrupted with one or two oxygen atoms, sulfur atoms, or a combination thereof.

7. The lipophilic anchor according to claim 1, wherein the anchor domain is derived from a second molecule comprising 2 to 6 hydrophobic tails.

8. The lipophilic anchor according to claim 6, wherein the second molecule comprises 2 or 3 hydrophobic tails.

9. The lipophilic anchor according to claim 1, wherein the linker domain is a structural unit generated upon reaction of the first molecule to the second molecule.

10. A lipophilic target molecule coated hydrophilic substrate, comprising:
a multiplicity of reaction residues of the functionality of the lipophilic anchor according to claim 1 and a complimentary functionality to the functionality of the conjugation domain of a lipophobic anchor on at least a hydrophilic surface of the hydrophilic substrate; and
a multiplicity of lipophilic target molecules non-covalently bound to the plurality of hydrophobic tails of the anchor domain.

11. The lipophilic target molecule coated hydrophilic substrate according to claim 10, wherein the hydrophilic surface comprises a hydrophilic polymer.

12. The lipophilic target molecule coated hydrophilic substrate according to claim 10, wherein the hydrophilic substrate comprises a hydrogel.

13. The lipophilic target molecule coated hydrophilic substrate according to claim 12, wherein the hydrogel is a dextran-based hydrogel.

14. The lipophilic target molecule coated hydrophilic substrate according to claim 10, wherein the multiplicity of lipophilic target molecules are selected from at least one biomolecule.

15. The lipophilic target molecule coated hydrophilic substrate according to claim 14, wherein the at least one biomolecule is selected from phosphatidylcholines (PC), phosphatidylethanolamines (PE), phosphatidylglycerols (PG), phosphatidylserines (PS), sterols, saccharolipids, lipopeptides, lipoproteins, and crude membrane components extracted from mammalian cells.

16. The lipophilic target molecule coated hydrophilic substrate according to claim 10, wherein the lipophilic target molecule is a lipid bilayer and the lipophilic target molecule coated hydrophilic substrate is a cell mimic.

17. A method of preparing the lipophilic anchor according to claim 1, comprising:
providing a first molecule comprising a multiplicity of functional groups selected from: acrylate, methacrylate, maleimide, vinylsulfone, aldehyde, acrylamide, vinyl, thiol, amine, alkylamine, hydroxy, derivatives of any of the foregoing, and any combination thereof;
providing a second molecule comprising at least one hydrophobic tail selected from saturated, mono-unsaturated, and polyunsaturated hydrocarbons and a multiplicity of complimentary functional groups;
combining the first molecule and the second molecule and optionally a solvent and/or a catalyst or an initiator; and
isolating the lipophilic anchor comprising the conjugation domain from the first molecule, the anchor domain from the second molecule, and the linker domain resulting from a reaction between the functional groups and the complimentary functional groups.

18. The method according to claim 16, wherein the functionality is an acrylate, and the complementary functionality is a thiol.

19. The method according to claim 16, wherein the optional catalyst is a trialkylamine.
